(19) United States
(12) Patent Application Publication
(10) Pub. No.: US 2017/0106021 A1
(43) Pub. Date: Apr. 20, 2017

(54) COMPOSITIONS ENRICHED FOR HOX11+ STEM CELLS AND METHODS OF PREPARING THE SAME

(71) Applicant: The General Hospital Corporation, Boston, MA (US)

(72) Inventor: Denise L. FAUSTMAN, Boston, MA (US)

(21) Appl. No.: 15/310,921

(22) PCT Filed: May 12, 2015
(86) PCT No.: PCT/US15/30282

§ 371 Date: Nov. 14, 2016

Related U.S. Application Data
(60) Provisional application No. 61/991,838, filed on May 12, 2014.

(51) Int. Cl. A61K 35/14; A61K 45/06; G01N 33/50; C12N 5/0789; C12Q 1/68; A61K 38/19; A61K 39/39

(52) U.S. Cl. CPC A61K 35/14; A61K 38/193; A61K 45/06; A61K 39/39; C12N 5/0647; C12Q 1/6876; G01N 33/5005; C12N 2506/11; C12N 2501/22; C12Q 2600/158

(57) ABSTRACT

The invention features enriched Hox11+ stem cell compositions and methods of preparing and using the same. In particular, the enriched Hox11+ stem cell composition can be used for treating medical conditions, diseases, or disorders, for transplantation and transplantation therapy, and for cellular, tissue, or organ repair or regeneration.

### BACKGROUND

Harvesting hematopoietic stem cells from peripheral blood with the administration of granulocyte-colony stimulating factor (G-CSF) to donors has advanced the field of hematopoietic stem cell transplantation. The use of G-CSF to mobilize stem cells to the peripheral blood for transplantation has replaced the direct harvesting of stem cells from bone marrow. Broad clinical data, such as a meta-analysis of nine large randomized clinical trials pooling more than 1,100 patients (Stem Cell Trialists' Collaborative Group, J. Clin. Oncol. 23:5074-5087, 2005), consistently confirm the advantages of peripheral blood stem cells transplants (PBST) for faster hematopoietic recovery, more circulating CD34+ and CD3+ cells in the blood and better clinical outcomes with reduced relapse rates (Anasetti et al., New Eng. J. of Med. 367:1487-1496, 2012). A disadvantage of PBST was a higher incidence of graft-versus-host disease (GVHD), presumably due to the simultaneous transplantation of mature T cells with the harvest. The further enrichment of CD34+ stem cells from G-CSF mobilized peripheral blood cells to produce a mature T cell-depleted mixture did not generate superior clinical benefit and in some cases, showed slower rates of diverse lymphocyte, B cell, and neutrophil recovery with increased incidences of bacterial infections (Bourhis et al., Haematologica 92:1083-1090, 2007).

The stem cells of the spleen are not commonly found in the peripheral blood or bone marrow of humans, but are vividly expressed in the spleen throughout a human's life (Dieguez-Acuña et al., Nature Leukemia 21:2192-2194, 2007). Adult human spleens harbor throughout life an early stem cell that expresses the Hox11 oncogene, also known as the Tlx1 gene. These stem cells of the spleen lack the mature CD45 marker of lymphoid cells or the immature CD34 proteins of hematopoietic lineage committed cells (Dieguez-Acuña et al., Int. J. Biochem. Cell Biol. 42:1651-1660, 2010; Dieguez-Acuña et al., Leukemia 21:2192-2194, 2007). In murine and rodent animal models, adult derived Hox11+ stem cells (sometimes referred to as Hox11+, CD45− stem cells) transferred into experimental hosts with end organ damage were able to aid the repair of inner ear, cranial neurons, heart tissue, pancreatic islet cells, bone morphogenesis, and salivary gland function, and contribute to hematopoietic cells (Lonyai et al, Hormone Metabolism Research 40:137-146, 2008). Developmental biologists have defined the Hox11+ stem cells to be involved in the genesis of the tissues that they help to repair (Dear et al., Development 121:2909-2915, 1995). Hox11+ stem cells in mouse embryogenesis first appear in the para-aortic splanchnopleura, a bank of tissue with the earliest site of hematopoiesis and with further development, migrate to the embryonic liver and spleen where they continue to generate hematopoietic stem cells until seeding of the bone marrow with stem cells expressing CD34 and CD45 cell-surface proteins that indicate hematopoietic lineage commitment of the stem cells. Even in adult human life, the spleen has a unique role in complete B cell differentiation and resistance to infections. Splenectomy results in accumulations of naïve B cells and reduction of memory B cells (Kruetzmann et al., Journal of Experimental Medicine 197:939-945, 2003; Mamani-Matsuda et al., Blood 111:4653-4659, 2008; Martinez-Gamboa et al., Clin. Immunol. 130:199-212, 2009).

There exists a need for methods to harvest and, in particular, to enrich, splenic Hox11+ stem cells for use in stem cell therapy and organ and tissue repair or regeneration.

### SUMMARY OF THE INVENTION

Methods of mobilizing Hox11+ stem cells to peripheral blood include the administration of one or more mobilization agents (e.g., G-CSF) to a donor prior to peripheral blood collection. Mobilized stem cells (e.g., Hox11+ stem cells) from the collected peripheral blood may be further enriched to produce a desired cell population that is enriched in, e.g., Hox11+ stem cells. The enrichment methods may include, e.g., a negative selection method that removes non-target cells (e.g., using one or more antibodies to cell-surface proteins on the non-target cells). The negative selection method may be used to remove cells having cell-surface protein markers of mature lymphocytes, such as, e.g., one or more of CD3, CD4, CD16, CD19, CD20, CD21, CD34, CD45, CD56, and T cell receptor. In particular, the negative selection method is used to remove CD45+ cells. The enrichment methods may also include, e.g., a positive selection method directed to target cells (e.g., using one or more antibodies to cell-surface proteins on the target cells). For example, a positive selection method may be used to enrich for target cells having cell-surface protein marker, such as CD34+ cells. A final cell population of the invention that is obtained after enrichment includes, e.g., a substantially homogeneous Hox11+ stem cells population (sometimes referred to as Hox11+, CD45− stem cells) or cell population that includes the combination of Hox11-expressing stem cells and CD34+ stem cells (e.g., in a ratio of about 1:9 to about 9:1). Also included in the invention are pharmaceutical compositions prepared for the final cell populations, which are substantially enriched with Hox11+ stem cells or that have a combination of Hox11-expressing stem cells and CD34+ stem cells (e.g., in a ratio of about 1:9 to about 9:1). The pharmaceutical compositions may be used for transplantation and/or in tissue and organ repair or regeneration.

In a first aspect of the invention, the invention features a method of preparing a pharmaceutical composition. The method includes: a) administering at least one mobilization agent to a subject; and b) preparing the pharmaceutical composition by collecting Hox11+ stem cells from peripheral blood of the subject, such that the composition includes a cellular component having at least 1% Hox11+ stem cells.

In some embodiments, the mobilization agent of the method is selected from the group consisting of granulocyte colony-stimulating factor (G-CSF), granulocyte-macrophage colony-stimulating factor (GM-CSF), stem cell factor (SCF), Fms-related tyrosine kinase 3 (flt-3) ligand, stromal cell-derived factor 1 (SDF-1), agonists of the chemokine (C—C motif) receptor 1 (CCR1), such as chemokine (C—C motif) ligand 3 (CCL3, also known as macrophage inflammatory protein-1α (Mip-1α)), agonists of the chemokine (C—X—C motif) receptor 1 (CXCR1) and CXCR2, such as chemokine (C—X—C motif) ligand (CXCL1), CXCL2 (also known as growth-related oncogene protein-β (Gro-β)), and CXCL8 (also known as interleukin-8 (IL-8)), agonists of CXCR4, such as CTCE-002, ATI-2341, and Met-SDF-1, Very Late Antigen (VLA)-4 inhibitor, TG-0054, plerixafor (also known as AMD3100), AMD3465, and any combination thereof. In particular, the mobilization agent is G-CSF. In some embodiments, the mobilization agent is administered in combination with one or more chemotherapy agents or immunostimulants (e.g., plerixafor).

In some embodiments, the preparation of the pharmaceutical composition includes the use of apheresis, such as leukapheresis.

In some embodiments, the preparation of the pharmaceutical composition involves removing non-Hox11+ stem cells from the composition, such as by use of an antibody. In one embodiment, the antibody is attached to a magnetic bead and the non-Hox11+ stem cells are separated from the Hox11+ stem cells using a magnet. In another embodiment, the antibody is attached to a fluorophore and the non-Hox11+ stem cells are separated from the Hox11+ stem cells using fluorescence-activated cell sorting (FACS).

In other embodiments, the preparation of the pharmaceutical composition involves enriching Hox11+ stem cells in the composition by removing CD45+ cells and/or CD34+ stem cells.

In some embodiments, the non-Hox11-stem cells are characterized by expression of one or more of cell-surface markers selected from the group consisting of CD3, CD4, CD16, CD19, CD20, CD21, CD34, CD45, CD56, and T cell receptor.

In all embodiments of the first aspect of the invention, the method further involves quantifying the number of Hox11+ stem cells in the composition, particularly in which the quantifying includes detecting the number of Hox11+ cells in the composition relative to the number of non-Hox11+ cells in the composition. For example, the Hox11+ stem cells may be detected using an intracellular protein or mRNA marker. In particular, the intracellular protein or mRNA marker may be selected from the group consisting of Hox11, Mad2L1, Minichromosome maintenance complex component 7 (Mcm7), Mcm8, POLD1, Hox11, DNA topoisomerase 1 (Top1), and Top2β. In some embodiments, quantifying of the number of Hox11+ stem cells in the composition includes using quantitative polymerase chain reaction (PCR). For example, the quantifying may include detecting Hox11+ stem cells using primers specific to the Hox11 gene.

In other embodiments, the non-Hox11+ cells are detected using a protein marker (e.g., an intracellular or extracellular protein) or mRNA marker that is not present on Hox11+ stem cells. In particular, the protein or mRNA marker may be detected by use of an antibody or by use of nucleic acid amplification. The protein or mRNA marker may be selected from one or more of the following Dhfr, Ercc1, Hprt1, Lap18, Mad1/1, Pola, Polr2e, Tdt, Topbp1, and Ung or a surface marker of mature lymphocytes selected from one or more of CD3, CD4, CD16, CD19, CD20, CD21, CD34, CD45, CD56, and T cell receptor.

In some embodiments, the composition of the invention includes Hox11+ stem cells and CD34+ stem cells. The ratio of the Hox11+ stem cells to the CD34+ stem cells may be 9:1 to 1:9.

In all embodiments of the first aspect of the invention, the cellular component of the pharmaceutical composition includes about 1-90% Hox11+ stem cells (e.g., the cellular component may include at least about 5%, 10%, 20%, 25%, 30%, 35%, 40%, 45%, 50%, 55%, 60%, 65%, 70%, 75%, 80%, or 85% Hox11+ stem cells). The cellular component of the pharmaceutical composition may be a substantially homogeneous Hox11+ stem cell population (e.g., having fewer than 30% non-Hox11+ stem cells, in particular, having fewer than 25%, 20%, 15%, 10%, 9%, 8%, 7%, 6%, 5%, 4%, 3%, 2%, or 1% on-Hox11+ stem cells).

In a second aspect, the invention features a pharmaceutical composition including a cell population that includes at least 1% Hox11+ stem cells (e.g., at least about 1-90% Hox11+ stem cells, such as about 5%, 10%, 20%, 25%, 30%, 35%, 40%, 45%, 50%, 55%, 60%, 65%, 70%, 75%, 80%, or 85% Hox11+ stem cells).

In other embodiments, the pharmaceutical composition includes Hox11+ stem cells and CD34+ stem cells. For example, the pharmaceutical composition includes a ratio of Hox11+ stem cells to CD34+ stem cells of 9:1 to 1:9.

In all embodiments of the second aspect of the invention, the pharmaceutical composition is made by the method of any one of the embodiments of the first aspect of the invention.

In all embodiments of the second aspect of the invention, the pharmaceutical composition includes one or more pharmaceutically acceptable carriers or excipients.

In a third aspect, the invention features a method of medical therapy that involves administering the pharmaceutical composition of any one of the embodiments of the second aspect of the invention to a subject in need thereof.

In some embodiments, the subject is in need of tissue or organ repair or regeneration. The tissue or organ is pancreas, salivary gland, pituitary gland, kidney, heart, lung, hematopoietic system, cranial nerves, heart, blood vessels including the aorta, olfactory gland, ear, nerves, structures of the head, eye, thymus, tongue, bone, liver, small intestine, large intestine, gut, lung, brain, skin, peripheral nervous system, central nervous system, spinal cord, breast, embryonic structures, embryos, or testes. In some embodiments, the tissue or organ is damaged or deficient.

In other embodiments, the subject has an autoimmune disease, a neurological disorder, cancer, an age-related disease, trauma, or acute radiation syndrome.

In some embodiments, the autoimmune disease is selected from Alopecia Areata, Ankylosing Spondylitis, Antiphospholipid Syndrome, Addison's Disease, Hemolytic Anemia, Hepatitis, Behcets Disease, Bullous Pemphigoid, Cardiomyopathy, Celiac Sprue-Dermatitis, Chronic Fatigue Immune Dysfunction Syndrome (CFIDS), Chronic Inflammatory Demyelinating Polyneuropathy, Churg-Strauss Syndrome, Cicatricial Pemphigoid, Limited Scleroderma (CREST Syndrome), Cold Agglutinin Disease, Crohn's Disease, Discoid Lupus, Essential Mixed Cryoglobulinemia, Fibromyalgia-Fibromyositis, Graves' Disease, Guillain-Barré Syndrome, Hashimoto's Thyroiditis, Hypothyroidism, Idiopathic Pulmonary Fibrosis, Idiopathic Thrombocytopenia Purpura (ITP), IgA Nephropathy, Insulin dependent Diabetes, Juvenile Arthritis, Lichen Planus, Lupus, Ménière's Disease, Mixed Connective Tissue Disease, Multiple Sclerosis, Myasthenia Gravis, Pemphigus Vulgaris, Pernicious Anemia, Polyarteritis Nodosa, Polychondritis, Polyglandular Syndromes, Polymyalgia Rheumatica, Polymyositis and Dermatomyositis, Primary Agammaglobulinemia, Primary Biliary Cirrhosis, Psoriasis, Raynaud's Phenomenon, Reiter's Syndrome, Rheumatic Fever, Rheumatoid Arthritis, Sarcoidosis, Scleroderma, Sjögren's Syndrome, Stiff-Man Syndrome, Takayasu Arteritis, Temporal Arteritis/Giant Cell Arteritis, Ulcerative Colitis, Uveitis, Vasculitis, Vitiligo, and Wegener's Granulomatosis, In particular, the autoimmune disease is insulin dependent diabetes (also known as type 1 diabetes).

In some embodiments, the neurological disorder is selected from Parkinson's disease, Alzheimer's disease, Amyotrophic lateral sclerosis (ALS), Huntington's disease, traumatic brain injury, and spinal cord injury.

In some embodiments, the cancer is selected from bladder cancer, pancreatic cancer, cervical cancer, lung cancer, liver cancer, ovarian cancer, colon cancer, stomach cancer, virally induced cancer, neuroblastoma, breast cancer, prostate cancer, renal cancer, leukemia, sarcoma, and carcinoma.

In some embodiments, the age-related disease is selected from a metabolic disorder, an inflammatory disorder, a cardiovascular disease, diabetes type 1, diabetes type 2, artherosclerosis, Alzheimer's disease, dementia, clinical depression, obesity, muscular dystrophy, sarcopenia, cachexia and osteoporosis.

In some embodiments, the subject is in need of, or has received, a cellular, tissue, or organ transplant. For example, the transplant may be a heart, heart valve, blood vessel (e.g., artery or vein), kidney, liver, lung, or lung lobe, pancreas, ovary, bladder, stomach, testis, intestine, thymus, bone, tendon, cornea, skin, nerve, hand, arm, foot, leg, beta-islet cell, or stem cell transplant.

In some embodiments, the Hox11+ stem cells in the composition are allogeneic or autologous to the subject.

A fourth aspect of the invention features a method of preparing a pharmaceutical composition containing Hox11+ stem cells. The method involves preparing the pharmaceutical composition by collecting Hox11+ stem cells from peripheral blood obtained from a subject administered at least one mobilization agent (e.g., G-CSF). In an embodiment, the composition includes a cellular component having at least 1% Hox11+ stem cells (e.g., at least about 1-90% Hox11+ stem cells, such as about 5%, 10%, 20%, 25%, 30%, 35%, 40%, 45%, 50%, 55%, 60%, 65%, 70%, 75%, 80%, or 85% Hox11+ stem cells). In other embodiments, the pharmaceutical composition includes Hox11+ stem cells and CD34+ stem cells. For example, the pharmaceutical composition includes a ratio of Hox11+ stem cells to CD34+ stem cells of 9:1 to 1:9.

### DEFINITIONS

As used herein, the term “Hox11+ stem cells” refers to stem cells expressing the Hox11 oncogene, also known as the Tlx1 gene. In the invention, Hox11+ stem cells may be mobilized from the spleen to the peripheral blood by administration of a mobilization agent (e.g., G-CSF). Hox11+ stem cells are characterized by the lack of expression of one or more, or all, of the following cell-surface markers: CD3, CD4, CD16, CD19, CD20, CD21, CD34, CD45, CD56, T cell receptor, and other mature lymphocyte markers. Hox11+ stem cells are sometimes referred to as “Hox11+, CD45− stem cells.”

As used herein, the term “apheresis” refers to a medical procedure in which the blood of a donor is circulated through an apparatus that separates out and harvests one particular constituent, e.g., a specific cell type, e.g., stem cells, such as Hox11+ stem cells and CD34+ stem cells, and returns the remainder of the blood to the circulation of the donor. Apheresis is commonly used to collect stem cells for autologous, syngeneic, and allogeneic transplantation at donation centers and hospitals. In some embodiments of the invention, peripheral blood of a donor treated with a mobilization agent (e.g., G-CSF) is returned to the donor through apheresis after the stem cells are collected. The term “leukapheresis” refers to a type of apheresis that can be used to separate white blood cells from the blood.

As used herein, the term “peripheral blood” or “mobilized peripheral blood” refers to peripheral blood that is obtained from a subject (e.g., a donor) that has been administered a mobilizing agent that enhances or increases the number of stem cells present in the peripheral blood prior to collection of the blood. The mobilization agent (e.g., G-CSF) may be used to increase the movement of stem cells that reside in the bone marrow (e.g., CD34+ stem cells), spleen (e.g., Hox11+ stem cells), or other organs to the peripheral blood.

As used herein, the term “enrich,” “enriched,” or “enrichment” refers to an increase in the percentage of one type of cell (e.g., a desired cell type, such as Hox11+ cells) in a final cell population (e.g., “an enriched cell population”) by at least about 0.01% over the percentage of the same type of cell in the starting (i.e., unenriched or initial) cell population. To enrich for one specific cell type relative to other cell types, a positive or negative selection method, or both may be used. For example, to produce a final cell population, desired cells may be isolated from a starting cell population (e.g., by targeting the desired cells), undesired cells may be removed from a starting cell population (e.g., by targeting the undesired cells), or both. Accordingly, a final cell population is considered “enriched” with respect to a desired cell type when the final cell population contains a higher percentage or number of the desired type of cells relative to the percentage or number of the desired type of cells in a starting population. The term “enriching,” which may be used synonymously with term “isolating.” Preferably, enriching produces a final cell population in which the percentage of one type of cell (e.g., Hox11+ stem cells) is increased by about 0.01%, 0.05%, 0.1%, 0.2%, 0.3%, 0.4%, 0.5%, 1%, 2%, 5%, or 10%, by about 20%, by about 30%, by about 40%, by about 50% or by greater than 50% as compared to the percentage of the one type of cell in a starting or initial population of cells.

As used herein, the term “positive selection” refers to a method whereby a desired cell type is targeted for selection, e.g., using an antibody to a specific cell-surface protein marker on the desired or target cell type.

As used herein, the term “negative selection” refers to a method whereby an undesired or non-target cell type is targeted for depletion or removal, e.g., using antibodies to specific cell-surface protein markers on the undesired or non-target cell type.

As used herein, the term “peripheral blood cells” refer to the cellular components of blood, including red blood cells, white blood cells, and platelets, which are found within the circulating pool of blood.

As used herein, the term “stem cells” refers to cells with the capacity or potential to differentiate to a more specialized or differentiated cell type, e.g., cells of a particular tissue or organ, and which retain the capacity under certain circumstances to proliferate without substantially differentiating. Stem cells are capable of proliferation and of giving rise to more undifferentiated or differentiated daughter cells. The daughter cells themselves can be induced to proliferate and produce progeny that subsequently differentiate into one or more mature and specialized cell types. Stem cells can be found in many tissues and organs of the body, such as bone marrow, spleen, and umbilical cord. In particular, Hox11+ stem cells (also referred to as Hox11+, CD45− stem cells) can be found in spleen and mobilized into peripheral blood using a mobilizing agent, such as G-CSF.

As used herein, the term “hematopoietic stem cell” refers to blood cells that have the capacity to self-replicate and to differentiate to precursors of blood cells. These precursor cells are immature blood cells that cannot self-replicate and must differentiate into mature blood cells. Hematopoietic stem cells display a number of phenotypes, such as Lin-CD34+CD38−CD90+CD45RA−, Lin-CD34+CD38−CD90−CD45RA−, Lin-CD34+CD38+IL-3aloCD45RA−, and Lin-CD34+CD38+CD10+(Daley et al., Focus 18:62-67, 1996; Pimentel, E., Ed., Handbook of Growth Factors Vol. III: Hematopoietic Growth Factors and Cytokines, pp. 1-2, CRC Press, Boca Raton, Fla., 1994). Within the bone marrow microenvironment, the stem cells self-proliferate and maintain continuous production of hematopoietic stem cells for all mature blood cells throughout life.

As used herein, the term “stem cells from the bone marrow” or “bone marrow-derived stem cells” refers to stem cells found in the bone marrow that can reconstitute the hematopoietic system, possess endothelial, mesenchymal, and pluripotent capabilities. Stem cells may reside in the bone marrow, either as an adherent stromal cell type, or as more differentiated cells that express CD34 or CD45 cell-surface protein, which indicates the stem cells' commitment to differentiate into blood cells.

As used herein, the term “marker” refers to a cell-surface or intracellular protein or nucleic acid molecule that serves as a distinct label to identify a certain cell type. Intracellular protein markers for Hox11+ stem cells include, e.g., one or more of mitotic arrest deficient-2 like-1 protein (Mad2L1), minichromosome maintenance complex component 7 protein (Mcm7), Mcm8, DNA polymerase Δ-1 catalytic subunit protein (Pold1), DNA topoisomerase-1 (Top1), and Top2b. Cell-surface protein markers for hematopoietic stem cells include one or more of the following: CD3, CD4, CD16, CD19, CD20, CD21, CD34, CD45, CD56, T cell receptor, and other markers of mature lymphocytes.

As used herein, the term “mobilization” or “stem cell mobilization” refers to a process involving the recruitment of stem cells from their tissue or organ of residence to peripheral blood following treatment with a mobilization agent, such as cytokines and chemotherapeutic drugs (e.g., G-CSF). This process mimics the enhancement of the physiological release of stem cells from tissues or organs in response to stress signals during injury and inflammation. The mechanism of the mobilization process depends on the type of mobilization agent administered. Some mobilization agents act as agonists or antagonists that prevent the attachment of stem cells to cells or tissues of their microenvironment. Other mobilization agents induce the release of proteases that cleave the adhesion molecules or support structures between stem cells and their sites of attachment.

As used herein, the term “mobilization agent” refers to a wide range of molecules that act to enhance the mobilization of stem cells from their tissue or organ of residence, e.g., bone marrow (e.g., CD34+ stem cells) and spleen (e.g., Hox11+ stem cells), into peripheral blood. Mobilization agents include chemotherapeutic drugs, e.g., cyclophosphamide and cisplatin, cytokines, and chemokines, e.g., granulocyte colony-stimulating factor (G-CSF), granulocyte-macrophage colony-stimulating factor (GM-CSF), stem cell factor (SCF), Fms-related tyrosine kinase 3 (flt-3) ligand, stromal cell-derived factor 1 (SDF-1), agonists of the chemokine (C—C motif) receptor 1 (CCR1), such as chemokine (C—C motif) ligand 3 (CCL3, also known as macrophage inflammatory protein-1α (Mip-1α)), agonists of the chemokine (C—X—C motif) receptor 1 (CXCR1) and CXCR2, such as chemokine (C—X—C motif) ligand (CXCL1), CXCL2 (also known as growth-related oncogene protein-β (Gro-β)), and CXCL8 (also known as interleukin-8 (IL-8)), agonists of CXCR4, such as CTCE-002, ATI-2341, and Met-SDF-1, Very Late Antigen (VLA)-4 inhibitor, TG-0054, plerixafor (also known as AMD3100), AMD3465, or any combination of the previous agents. A mobilization agent increases the number of stem cells in peripheral blood, thus allowing for a more accessible source of stem cells for use in transplantation, organ repair or regeneration, or treatment of disease.

As used herein, the term “splenic stem cell” refers to stem cells derived from the spleen, e.g., Hox11+ stem cells (also referred to as Hox11+, CD45− stem cells).

### DESCRIPTION OF THE DRAWINGS

FIGS. 1A to 1D show bar graphs depicting the relative quantity of CD34 and Hox11 mRNA expressions in peripheral blood lymphocytes (PBLs) obtained from non-mobilized (non-G-CSF treated, n=10) and mobilized (G-CSF treated, n=18) peripheral blood samples. (A) and (B) These sections represent the mean of CD34 or Hox11 mRNA expressions of non-mobilized and mobilized PBLs. (C) and (D) These sections represent the individual data points of the donors used for the data in (A) and (B).

FIGS. 2A to 2D show bar graphs depicting the relative quantity of CD34 and Hox11 mRNA expressions in G-CSF mobilized PBLs with or without further enrichment of CD34+ stem cells. (A) and (B) These sections represent the mean of CD34 or Hox11 mRNA expressions in G-CSF mobilized PBLs with or without CD34 enrichment. (C) and (D) These sections represent the individual data points of the donors used for the data in (A) and (B). n=18 for G-CSF mobilized PBLs without CD34 enrichment. n=4 for G-CSF mobilized PBLs with CD34 enrichment.

### DETAILED DESCRIPTION OF THE INVENTION

Splenomegaly and splenic rupture (Veerappan et al., Bone Marrow Transplantion 40:361-364, 2007; Stroncek et al., Journal of Translational Medicine 2:25-28, 2004) are common complications of G-CSF administration to obtain mobilized peripheral blood. I recognized that these complications reveal a role of the spleen in G-CSF treated stem cell mobilization and in stem cell transplantation. In particular, I discovered that stem cells expressing the Hox11 gene (herein referred to as “Hox11+ stem cells”), which are normally present in the spleen, can be obtained in mobilized peripheral blood following the administration of a mobilization agent (e.g., G-CSF).

The lack of splenic stem cell visibility in the blood has hindered clinical practice since sampling the spleen is not an easy or necessarily safe surgical method that could be routinely used. Accordingly, the methods and compositions disclosed herein provide enriched splenic Hox11+ stem cell compositions. In particular, the methods produce a population of cells from peripheral blood that is substantially enriched for Hox11+ stem cells (e.g., including compositions that are substantially homogeneous for Hox11+ stem cells). The methods may include preparing cell compositions by removing other non-Hox11+ stem cells, such as CD45+ and/or CD34+ cells. Alternatively, the methods may involve producing a composition having a population of cells that includes Hox11+ stem cells and CD34+ cells in a ratio of, e.g., 1:9 to 9:1. These methods and compositions have potential therapeutic applications in stem cell-related transplants, stem cell therapy, and tissue and organ repair and regeneration.

### Stem Cell Mobilization

Stem cells reside in the bone marrow and various organs of the body, such as umbilical cord, liver, spleen, heart, and lung. In general, under normal conditions in a healthy human body, only a very low number of stem cells is found circulating in peripheral blood. Hox11+ stem cells, for example, are normally not found in peripheral blood. Hematopoietic stem cells, which normally reside in the bone marrow, account for only 0.01-0.05% of cells in peripheral blood. Historically, to extract stem cells for autologous or allogeneic stem cell transplantation, multiple needle aspirations were utilized to obtain stem cells from the bone marrow of the pelvic bone, an extremely complicated and risky procedure. Later studies showed that stem cells residing in the bone marrow and other organs of the body can be stimulated by certain growth factors, cytokines, chemokines, and chemotherapeutic drugs to detach from their microenvironments and enter into peripheral blood circulation. In recent years, bone marrow stem cell collection has been largely replaced in favor of peripheral blood stem cell collection, which is faster, safer, and has fewer major complications compared to bone marrow stem cell collection. Transplantations performed using stem cells harvested from peripheral blood demonstrated more robust hematopoietic engraftment and lower mortality from complications. Currently, nearly all autologous stem cell transplantation and the majority of allogeneic stem cell transplantation are performed using circulating peripheral blood stem cells (Hemchandra et al., Frontiers in Biosci. 4:611-619, 2012).

Hox11+ stem cells in the spleen can be mobilized to peripheral blood by administering one or more growth factors, cytokines, chemokines, and chemotherapeutic drugs, which are collectively called mobilization agents, to donors. These mobilization agents interfere with attachment or adhesion of Hox11+ stem cells to their resident microenvironment, leading to increased release of these stem cells into peripheral blood circulation. For example, some mobilization agents target chemokine receptors and adhesion factors, e.g., CXCR4 and VLA4 antagonists, disrupt Hox11+ stem cell adhesion to cells in the spleen, and alter the chemotactic gradients in the microenvironment. Other mobilization agents, such as G-CSF or the chemotherapeutic agent cyclophosphamide, create a highly proteolytic environment in the organ by inducing the release of a number of proteases, such as metalloproteinase-9 (MMP-9), cathepsin G, and neutrophil elastase, that in turn cleave a variety of Hox11+ stem cell supportive molecules.

To obtain peripheral blood samples containing Hox11+ stem cells clinically, donors are treated with one or more mobilization agents prior to donating blood. Examples of mobilization agents include, but are not limited to, granulocyte colony-stimulating factor (G-CSF), granulocyte-macrophage colony-stimulating factor (GM-CSF), stem cell factor (SCF), Fms-related tyrosine kinase 3 (flt-3) ligand, stromal cell-derived factor 1 (SDF-1), agonists of the chemokine (C—C motif) receptor 1 (CCR1), such as chemokine (C—C motif) ligand 3 (CCL3, also known as macrophage inflammatory protein-1α (Mip-1α)), agonists of the chemokine (C—X—C motif) receptor 1 (CXCR1) and CXCR2, such as chemokine (C—X—C motif) ligand 1 (CXCL1), CXCL2 (also known as growth-related oncogene protein-β (Gro-β), and CXCL8 (also known as interleukin-8 (IL-8)), agonists of CXCR4, such as CTCE-002, ATI-2341, and Met-SDF-1, Very Late Antigen-4 (VLA-4) inhibitor, TG-0054, plerixafor (also known as AMD3100), AMD3465, cyclophosphamide, cisplatin, and combinations thereof. Both G-CSF and GM-CSF are FDA approved for stem cell mobilization in healthy donors. Compared to G-CSF, GM-CSF may mobilize fewer Hox11+ stem cells and may require that donors participate in more leukapheresis sessions for adequate stem cell collection.

The combination of G-CSF and plerixafor is FDA approved for stem cell mobilizations in patients with non-Hodgkin's lymphoma and multiple myeloma. G-CSF is the predominant cytokine used in most transplant centers. Hox11+ stem cells can be mobilized using one or more of clinically available mobilization agents, which are described in detail below.

Mobilization with G-CSF or GM-CSF

G-CSF can be used to mobilize Hox11+ stem cells into peripheral blood. In some examples of Hox11+ stem cell mobilization, G-CSF may be administered daily as a dose of 0.5-16 μg/kg (e.g., 5-16 μg/kg or 10-16 μg/kg) for 1-10 days (e.g., 1-7 days, or particularly, 1-3 days). In another example, G-CSF may be administered to a healthy donor at a dosage of 10-16 μg/kg daily for up to seven days. Three or four days of treatment may be sufficient when the peripheral blood collections are combined with apheresis starting on, e.g., day 4. In another example, G-CSF may be administered at a dosage of 10 μg/kg daily for four days with apheresis starting on, e.g., day 4. The most commonly used dosage of G-CSF in healthy donors is 10 μg/kg body weight daily with leukapheresis starting on day 5 onward until collection of an adequate number of stem cells (e.g., collections can be taken once or twice daily for 1 to 4 days, such as 1 or 2 days). Twice daily administration of G-CSF may mobilize more Hox11+ stem cells than a single daily dosage of G-CSF (Kroger et al., Br. J. Haematol. 111:761-765, 2000; Engelhardt et al., J. Clin. Oncol. Jul. 17:2160-2172, 1999). G-CSF may be administered subcutaneously or intravenously. Methods of G-CSF administration and dosage are described in Juttner et al. (Blood 89:2233-2258, 1997), Kroschinsky et al. (Haematologica 90:1556-1671, 2005), U.S. Pat. No. 6,162,427, 2005/0186182, WO 2010051335, and WO 2005014023, all of which are incorporated herein by reference in their entireties.

Besides being used as a single mobilization agent, G-CSF may be administered alone or in combination with one or more other mobilization agents and/or one or more chemotherapy agents, such as cyclophosphamide, to mobilize Hox11+ stem cells. If cyclophosphamide is administered for mobilization, G-CSF is usually started 2-5 days after completion of cyclophosphamide infusion. Methods of administering combined mobilization agents of G-CSF and one or more chemotherapeutic agents are described in Andre et al. (Transfusion 43:50-57, 2003), Ataergin et al. (Am. J. Hematol. 83:644-648, 2008), and Demirer et al. (Br. J. Haematol. 116:468-474, 2002), all of which are incorporated herein by reference in their entireties.

One or more sessions (e.g., 1-4 sessions) of apheresis may be needed to collect an adequate number of Hox11+ stem cells. The number of apheresis sessions depend on, for example, the volume of G-CSF mobilized peripheral blood collected at each aphesis session, whether G-CSF is continued to be administered during apheresis sessions, as well as whether the donor is a robust or poor mobilizer. The number of Hox11+ stem cells collected from a donor may vary from ˜1×10⁵ to ˜10×10⁶ cells.

Mobilization with Plerixafor or Plerixafor in Combination with G-CSF

Plerixafor (AMD3100) is a bicyclam molecule that reversibly inhibits CXCL12 binding to CXCR4, disrupting the adhesion of stem cells to their microenvironment. Plerixafor can be administered alone or in combination with another mobilization agent, such as G-CSF, to mobilize Hox11+ stem cells into peripheral blood. Plerixafor may be administered at a dosage of 1-300 μg/kg. At 240 μg/kg, the number of mobilized stem cells peak at around 4-10 hours after plerixafor administration. In some examples of Hox11+ stem cell mobilization, plerixafor may be administered once or twice daily at a dose of ˜1-300 μg/kg (e.g., 100-300 μg/kg or 200-300 μg/kg) for 1-10 days (e.g., 1-5 days).

The combination of plerixafor and G-CSF for stem cell mobilization was approved by the FDA in 2008 for use in patients with non-Hodgkin's lymphoma and multiple myeloma. This combination therapy may also be used to mobilize Hox11+ stem cells. A typical combination therapy may include, e.g., the administration of G-CSF at ˜0.5-16 μg/kg (e.g., 10 μg/kg) daily with plerixafor at 1-300 μg/kg (e.g., 240 μg/kg) given a few days (e.g., 1-3 days) after G-CSF administration. Both agents may be given together for about 2-10 days (e.g., 4 days) or until adequate Hox11+ stem cells are collected. Plerixafor, either alone or in combination with G-CSF, may be administered subcutaneously or intravenously.

To collect Hox11+ stem cells from plerixafor or plerixafor and G-CSF mobilized peripheral blood, one or more sessions (e.g., 1, 2, 3, 4, 5, 6, 7, 8, 9, or 10 sessions) of apheresis (e.g., once or twice daily) may be used to collect an adequate number of Hox11+ stem cells. In some examples, the most optimal time to start the first apheresis session may be at around 4-10 hours after plerixafor administration. The number of apheresis sessions depend on, for example, the volume of plerixafor mobilized peripheral blood collected at each aphesis session, whether G-CSF is to be co-administered with plerixafor during apheresis sessions, and whether the donor is a robust or poor mobilizer. The number of Hox11+ stem cells collected may vary from ˜1×10⁵ to ˜10×10⁶ cells.

Overall, mobilization agents for Hox11+ stem cell mobilization often differ in their mode of administration, the time needed to achieve mobilization, and their efficiency. In some examples, donors receiving intensive chemotherapy and older donors may be poor mobilizers, and thus may require longer term administration of mobilization agents or more collection sessions. In particular, these donors are often treated with higher doses of a mobilization agent, such as G-CSF, GM-CSF, G-CSF with GM-CSF, G-CSF with plerixafor, or other combination of mobilization agents and chemotherapy drugs described herein.

### Hox11+ Stem Cell Donors

A preferred donor of Hox11+ stem cells is a healthy individual. Hox11+ stem cells isolated from mobilized peripheral blood of a healthy donor may be used in, e.g., allogeneic transplantations. Hox11+ stem cells may also be collected from mobilized peripheral blood of a healthy donor for use in organ repair or regeneration therapies or other therapies described herein in related or unrelated recipients.

In other examples, a donor may be in poor health and/or one that is undergoing treatment (e.g., receiving one or more chemotherapeutic agents) for certain diseases (e.g., cancer). Hox11+ stem cells may be isolated from mobilized peripheral blood of an unhealthy donor for their own use or for use in related or unrelated recipients. For example, Hox11+ stem cells isolated from mobilized peripheral blood of diseased donors may be used in autologous or allogeneic transplantations or in organ repair or regeneration therapies or other therapies described herein in related or unrelated recipients.

Stem Cell Collection from Peripheral Blood

The ability to collect and purify populations and subpopulations of stem cells is fundamental to their use in autologous and allogeneic therapies (e.g., transplantation or for use in organ repair or regeneration therapies). Populations of cells for use in the methods described herein (e.g., cell compositions that contain Hox11+ stem cells or Hox11 stem cells and CD34+ stem cells) may be mammalian cells, such as human cells, non-human primate cells, rodent cells (e.g., mouse or rat), bovine cells, ovine cells, porcine cells, equine cells, sheep cell, canine cells, and feline cells or a mixture thereof. The cells may be obtained from an animal, e.g., a human patient, or they may be from cell lines. If the cells are obtained from an animal, they may be subjected to purification methods and used as, e.g., a substantially enriched population (e.g., a substantially enriched population of Hox11+ stem cells) or as a mixed population (e.g., a mixed population of Hox11+ stem cells and CD34+ stem cells). The cells may also be expanded by culturing after collection from an animal (described below).

To purify and enrich a population of Hox11+ stem cells, the target cells may be separated from other components in blood (e.g., peripheral blood), such as platelets, plasma proteins, and non-target cells (e.g., CD45+ cells). The target cell population may be collected from one or more subjects that have been administered one or more mobilization agents, such as G-CSF. The source of the cell population may be blood, e.g., peripheral blood. The blood may be collected from the subject (the “donor”) without separation of any blood components or it may be processed prior to or after collection from the subject in order to remove undesired blood components (e.g., platelets, plasma proteins, and non-target cells (e.g., CD45+ cells)). A suitable volume of a blood sample could be from about 1 to about 50 ml, e.g., about 5 to 50 ml, about 5 to 15 ml, or more specifically, about 1, 2, 3, 4, 5, 6, 7, 8, 9, 10, 11, 12, 13, 14, 15, 16, 17, 18, 19, 20, 25, 30, 35, 40, 45, or 50 ml or any range derivable therein. The cell population may also be obtained from a cryopreserved blood sample. Alternatively, a composition that includes the target cell population (e.g., Hox11+ stem cells) in purified (e.g., enriched or separated from non-target cell components, such as CD45+ cells) or semi-purified form (e.g., not yet fully enriched or separated from non-target cell components) may be cryopreserved.

Mobilized peripheral blood samples can be collected from the bloodstream of donors (e.g., via intravenous (IV) collection) using any one of several known methods. One method is to pass the blood through an apheresis machine. Apheresis is a procedure or process in which peripheral blood drawn from a donor is separated into its various blood components, such as plasma, platelets, and/or cells (e.g., stem cells); the desired blood components (e.g., Hox11+ stem cells) are collected and the remainder of the blood components are returned to the donor. Another similar method is leukopheresis. A review of peripheral blood stem cell collection can be found in Shpall et al. (Annu. Rev. Med. 48:241-251, 1997), which is incorporated herein by reference.

Following apheresis or leukapheresis, the cells present in the sample may be counted using well-known techniques or instruments in the art. Methods for cell counting are described in Brando et al. (Cytometry 42:327-346, 2000), Siena et al. (Blood 77:400-409, 1991), Chapple et al. (Cytotherapy 2:371-376, 2000), Gratama et al. (J. Biol. Regul. Homeost. Agents 15:14-22, 2001), U.S. Pat. No. 3,406,121, WO 2012166952, and WO 2011091007, all of which are incorporated herein by reference in their entireties.

Once mobilized peripheral blood samples are obtained, the target cells (e.g., Hox11+ stem cells) can be separated from non-target cells (e.g., CD45+ cells) that may be present in the collected blood samples using any one or more of several methods in order to produce a substantially enriched Hox11+ stem cell composition.

An enriched Hox11+ stem cell population may include at least, about, or at most, 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, or 10×10⁶ Hox11+ stem cells or any range derivable therein.

Optionally, the enriched Hox11+ stem cell population may be expanded ex vivo by culturing them in the presence of agents that stimulate proliferation of Hox11+ stem cells. Methods of ex vivo stem cell expansion from peripheral blood are known in the art (see, for example, Bruggar et al., Blood 81:2579-2584, 1993, which is incorporated herein by reference). In certain aspects, starting cells prior to expansion may include at least or about 10³, 10⁴, 10⁵, 10⁶, 10⁷, 10⁸, 10⁹, 10¹⁰, 10¹¹, 10¹², 10¹³ cells or any range derivable therein. The starting cell population may have a seeding density of at least or about 10, 10², 10³, 10⁴, 10⁵, 10⁶, 10⁷, 10⁸ cells/ml, or any range derivable therein. The methods may also include expanding stem cells in the collected peripheral blood (e.g., Hox11+ stem cells) prior to therapeutic use. In other embodiments, the cell population may be essentially free of any terminally differentiated blood cells, like T cells or B cells (e.g., CD45+ cells).

### Methods and Techniques to Provide an Enriched Hox11+ Stem Cell Subpopulation

Once the stem cells are collected from peripheral blood, various methods in the art can be used to select and enrich for Hox11+ stem cells in the final cell population. Available methods can be grouped into two main categories: techniques based on antibody capture that utilize cell-surface markers and techniques that separate stem cells based on their physical properties, such as size, density, volume, diffusivity, and/or surface charge.

Peripheral blood cells may also be collected from peripheral blood using leukapheresis, which is a specific type of apheresis in which white blood cells are separated from peripheral blood. Once a population of cells is collected from the peripheral blood sample, the cells can undergo further enrichment (described in detail below) to increase the percentage of Hox11+ stem cells. For example, the cell population may then be enriched for Hox11+ stem cells using negative selection to remove CD45+ cells or CD45+ and CD34+ cells. For further enrichment of Hox11+ stem cells, other cells, e.g., those expressing markers of mature lymphocytes, such as CD3, CD4, CD16, CD19, CD20, CD21, CD34, CD45, CD56, and T cell receptor, may also be targeted for removal.

Antibody capture techniques may be used in a negative selection method to enrich for Hox11+ stem cells in the cell population. It is generally recognized in the field that most of the cells in peripheral blood express the cell-surface protein marker CD45, while Hox11+ stem cells lack the CD45 cell-surface protein. Hox11+ stem cells are sometimes referred to as Hox11+, CD45− stem cells. In a negative selection method, antibodies that target the CD45 surface marker may be used to capture and remove CD45+ cells, thereby producing an enriched population of Hox11+ stem cells. The enriched population may also include CD34+ stem cells (which also do not express CD45 cell-surface protein).

Depending on the therapeutic application, one may wish to administer a population of cells that includes Hox11+ stem cells and CD34+ stem cells. In some examples, CD45+ cells and CD34+ stem cells may be removed so as to produce a cell population that is substantially enriched for Hox11+ stem cells. For example, antibodies that target the CD34 surface marker may be used to capture and remove CD34+ stem cells, thereby producing an enriched population that is substantially homogeneous for Hox11+ stem cells only. Therefore, the resultant cell population contains low to no concentration of undesired cells (e.g., CD45+ cells), and consequently a higher concentration of the desired Hox11+ stem cells. After CD45+ cells are removed, the remaining cells could be used for clinical re-infusion practices—not only for hematopoietic reconstitution but also for healing of end organs, such as blood, inner ear, spinal ganglia, pituitary gland, kidney, mammary gland, olfactory gland, bone, pancreas, testes, salivary glands, liver, brain, peripheral nervous system, central nervous system, spinal cord, and heart. Moreover, compositions that are enriched for Hox11+ stem cells obtained from peripheral blood may also provide better engraftment and may reduce or eliminate the risk of graft-versus-host disease (GVHD), which is often caused by the higher T-cell content in non-purified peripheral blood cell populations.

Target or non-target cells can be separated and removed from the total cell population using methods well-known in the art, e.g., antibody-based methods, such as fluorescence activated cell sorting (FACS). Commercial kits containing cell-type specific isolation reagents are also available. For example, MACS® Cell Separation Reagents from Miltenyi Biotec, which contain antibodies specific to cell-surface proteins conjugated to micro-magnetic beads, can be used to separate, e.g., T cells, B cells, cancer stem cells, and hematopoietic stem cells. Such methods typically isolate one stem cell population at a time from the peripheral blood sample. In some examples CD45+ cells may be particularly targeted for removal using, e.g., antibodies that bind to the cell-surface CD45 protein. The CD45-specific antibodies may be attached to fluorophores such that CD45+ cells may be separated using FACS. In other examples, CD45-specific antibodies may be attached to magnetic beads, e.g., CD45 MicroBead Kit from Miltenyi Biotec, such that CD45+ cells may be separated using magnets. In yet other examples, CD45-specific antibodies may be attached to secondary antibodies which can bind to immobilized antigens. Similar procedures can be applied to remove other cells with specific cell-surface proteins, e.g., CD34+ stem cells. Methods and techniques of antibody-based cell capture are known in the art. Such techniques are described in, e.g., Patent Publication Nos. WO 2001006254, WO 2010078872, WO 2013087234, and US 20130316373, each of which is incorporated herein by reference in its entirety.

In general, a primary antibody for positive selection methods may be used at a concentration from about 0.1 μg to about 10 μg per 10⁶ of targeted cells. The primary antibody is often attached to a solid support. Some examples of solid supports include membranes, surfaces, beads, resins, magnets, and particles. For example, primary antibodies attached to magnetic beads may be incubated with a population of cells that is present in a peripheral blood sample obtained, e.g., through apheresis or leukapheresis. The undesired, non-Hox11 expressing stem cells may be captured in a complex with the primary antibody and magnetic beads. The complex may then be separated from the remainder of the cell population using magnets. The magnetic bound cells may then be discarded. Commercial antibody selection technology is also available, such as Isolex 300i (Baxter Healthcare Corp), U.S. Pat. Nos. 5,536,475; 6,251,295; 5,968,753; and 6,017,719, all of which are incorporated herein by reference in their entireties.

Aside from antibody-based techniques, techniques that separate cells based on their physical properties, such as size, density, volume, diffusivity, and surface charge may also be used to produce an enriched Hox11+ stem cell population from mobilized peripheral blood. These techniques include, but are not limited to, solid phase cell isolation (U.S. Patent Publication No.: 2014/0045208), electrorotation (Yang et al., Biophysical Journal 76:3307-3314, 1999), elutriation (WO 2011/069117), and field flow fractionation (Giddings, Science 260:1456-1465, 1993). Techniques described in these references are incorporated herein by reference in their entireties. In brief, these techniques separate populations of cells from peripheral blood by subjecting the cells to different electric fields, e.g., rotational electric field, flow velocity rates, and centrifugal forces. Since various types of cells differ in their size, density, cell membrane capacity, and cell-surface charge, they experience different forces in the electric field or generate different retention rates in a liquid flow field. For example, electrorotation has been used to separate erythroleukemia cells from erythrocytes (Huang et al., Phys. Med. Biol. 40:1789-1806, 1995) by subjecting the cells to a rotational electric field. These techniques can also be used to produce a population of cells enriched for Hox11+ stem cells.

### Assay to Identify and Quantify Cells in an Enriched Hox11+ Stem Cell Population

After enrichment of Hox11+ stem cells, the final stem cell population can be evaluated for the presence and quantity of Hox11+ stem cells using various biochemical and molecular genetic techniques.

To directly identify and quantify Hox11+ stem cells, a subset of the cells may be separated and used for testing. Cells in the enriched Hox11+ stem cell population can be evaluated by the expression of DNA or RNA of cell-type specific genes. For example, quantitative mRNA expression analysis by reverse transcription polymerase chain reaction (RT-PCR) (Bustin, Journal of Molecular Endocrinology 25:169-193, 2000) can be used to characterize gene expression of intracellular proteins of Hox11+ stem cells. Compared to detection of protein expression, mRNA detection using RT-PCR is more sensitive, which is advantageous especially when the sample contains a limited number of cells.

For example, an aliquot of cells from the enriched Hox11+ stem cell population may be assessed for expression of the Hox11 gene and/or the CD34 gene, e.g., using quantitative mRNA expression analysis. Cells are lysed and mRNA is extracted using standard techniques known in the art. The purified mRNA is reverse transcribed to complementary DNA (cDNA) using standard techniques, e.g., High Capacity cDNA Reverse Transcription Kit (Applied Biosystems, Foster City, Calif.). Subsequently, quantitative polymerase chain reaction (PCR) may be performed using gene-specific primers.

Additionally, Hox11+ stem cells may also be permeabilized or lysed to give access to intracellular protein markers. For example, enriched Hox11+ stem cells can be fixed on a cell culture plate using crosslinking agents well known in the field, e.g., paraformaldehyde. Following cell fixation, the cell membrane can be permeabilized using organic solvents, e.g., methanol or acetone, or detergents, e.g., TritonX-100 or Tween-20 other. Cell permeabilization is a commonly practiced technique in the laboratory and can be used to provide antibody access to intracellular proteins. After cell permeabilization, cells can be incubated with specific antibodies to detect one or more intracellular protein markers of Hox11+ stem cells, including, but not limited to, Hox11, mitotic arrest deficient-2 like-1 protein (Mad2L1), minichromosome maintenance complex component 7 protein (Mcm7), Mcm8, DNA polymerase Δ-1 catalytic subunit protein (Pold1), DNA topoisomerase-1 (Top1), and Top2b. One or more of these markers may be detected, e.g., using marker-specific antibodies, after cell fixation and permeabilization.

In addition to antibody detection of intracellular proteins on fixed cells, intracellular proteins of Hox11+ stem cells can also be extracted, purified, and analyzed using commonly used techniques in the art, such as immunoblot and protein chromatography.

The presence of other stem cells in the enriched Hox11+ stem cell population (e.g., CD34+ stem cells) can also be determined using the above techniques or by detection of cell-surface protein markers. Cell-surface protein markers can be evaluated without the need for cell permeabilization or lysis. Antibody-based procedures can be used to detect the cell-surface proteins. The cells can be analyzed using flow cytometry, e.g., FACS, or observed under a fluorescence microscope after immunostaining. For example, after negative selection to remove CD45+ cells and/or CD34+ stem cells, the cells in the desired, final cell population should not stain positive for CD45 and/or CD34 protein markers. In this case, antibody detection of cell-surface protein markers may be used to confirm the absence of such cells in the enriched, Hox11+ stem cell population.

### Pharmaceutical Compositions and Preparations

Pharmaceutical compositions of the invention contain either a substantially enriched population of Hox11+ stem cells (e.g., lacking CD34+ cells and CD45+ cells) or a substantially enriched population of cells that includes Hox11+ stem cells and CD34+ stem cells, e.g., in a ratio of 1:9 to 9:1 (e.g., 1:9, 1:8, 1:7, 1:6, 1:5, 1:4, 1:3, 1:2, 1:1, 2:1, 3:1, 4:1, 5:1, 6:1, 7:1, 8:1, or 9:1). In addition to a therapeutic amount of stem cells, the pharmaceutical compositions may contain a pharmaceutically acceptable carrier or excipient, which can be formulated by methods known to those skilled in the art. The pharmaceutically acceptable carrier or excipient may be non-naturally occurring.

The pharmaceutical compositions of the invention may contain an enriched (e.g., substantially homogeneous) Hox11+ stem cell population (e.g., lacking CD34+ cells and CD45+ cells) or a combination of Hox11+ stem cells and CD34+ stem cells, in which the cell population is collected from peripheral blood of a subject administered one or more mobilization agents (e.g., G-CSF). The cell population in the pharmaceutical compositions may be further enriched for Hox11+ stem cells, e.g. by negative selection, such as by removing CD45+ cells or both CD34+ stem cells and CD45+ cells. In some examples, the cell population in the pharmaceutical compositions contain at least 1% Hox11+ stem cells (e.g., but without limitation to, at least or about 5%, at least or about 10%, at least or about 15%, at least or about 20%, at least or about 25%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 96%, at least or about 97%, at least or about 98%, or at least or about 99% Hox11+ stem cells). In some examples, the cell population in the pharmaceutical composition contains a ratio of Hox11+ stem cells to CD34+ stem cells that is in a range from 1:9 to 9:1. In other examples, the pharmaceutical composition contains a number of Hox11+ stem cells within the range of 500,000 to 50,000,000 per ml (e.g., at least about 1×10⁶ Hox11+ stem cells/ml) of the pharmaceutical composition.

For an enriched Hox11+ stem cell composition of the invention, it is preferred that the percentage of Hox11+ stem cells in the composition is greater than the percentage of Hox11+ stem cells in an unenriched cell population. Alternatively, an enriched Hox11+ stem cell composition of the invention may contain a greater percentage of Hox11+ stem cells than that which is naturally present in the peripheral blood of a subject not administered a mobilization agent (e.g., G-CSF). In still another embodiment, an enriched Hox11+ stem cell composition of the invention may contain a greater percentage of Hox11+ stem cells than that which is present in the peripheral blood of a subject that has been administered a mobilization agent (e.g., G-CSF).

Methods of producing pharmaceutical compositions containing stem cells for transplantation and regenerative medicine are described in, e.g., U.S. Pat. Nos. 8,465,733 and 8,652,846, and U.S. Patent Application Publication Nos. 2013/0209422, 2012/0301538, 2011/0076256, and 2013/0302293, which are incorporated herein by reference in their entireties.

Pharmaceutical compositions of the invention that contain an enriched population of Hox11+ stem cells may be stably stored at a temperature within a range of −80 to 25° C. for a period of time within a range of 1 hour to at least 30 days without significantly reducing the overall viability and functionality of the stem cells. Alternatively, an enriched population of Hox11+ stem cells may be stored under cryopreservation at a temperature of less than −196° C. for a period of time within a range of 30 days to 10 years (e.g., 1-5 years) without significantly reducing the overall viability and functionality of the stem cells. When ready for use, the cell composition can be thawed and prepared for administration. Cell viability can be determined by any means known in the art (e.g., the methods disclosed in Stoddart (Methods Mol. Biol. 740:1-6, 2011), Gerets et al. (Methods Mol. Biol. 740:91-101, 2011), and Petty et al. (Comparison of J. Biolum. Chemilum. 10:29-34, 1995)). Assays for cell viability are also available commercially, e.g., CELLTITER-GLO®Luminescent Cell Viability Assay (Promega), which uses luciferase technology to detect ATP and quantify the health or number of cells in culture, and the CellTiter-Glo® Luminescent Cell Viability Assay, which is a lactate dehyrodgenase cytotoxicity assay (Promega). Techniques and methods used to assess cell viability are described in U.S. Pat. Nos. 5,314,805, 5,185,450, 4,734,372, and in U.S. Patent Application Publication No. 2013/0210063, which are incorporated herein by reference in their entireties.

Pharmaceutical compositions of the invention can be administered parenterally in the form of an injectable formulation. Pharmaceutical compositions for injection can be formulated using a sterile solution or any pharmaceutically acceptable liquid as a vehicle. Pharmaceutically acceptable vehicles include, but are not limited to, sterile water, physiological saline, and cell culture media (e.g., Dulbecco's Modified Eagle Medium (DMEM), α-Modified Eagles Medium (α-MEM), F-12 medium). The pharmaceutical composition may also contain stabilizers, carriers, or excipients, such as human serum albumin (HSA), plasma proteins, dextran, glucose, D-sorbitol, D-mannose, D-mannitol, and sodium chloride, that act to stabilize or maintain the health and function of the enriched Hox11+ stem cells in the pharmaceutical composition.

The pharmaceutical composition may be formed in a unit dose form as needed. The amount of active component, e.g., Hox11+ stem cells, included in the pharmaceutical preparations is such that a suitable dose within the designated range is provided (e.g., a dose within the range of 500,000 to 80,000,000 Hox11+ stem cells per ml, such as at least about 1×10⁶ Hox11+ stem cells/ml).

### Routes, Dosage, and Timing of Administration

Pharmaceutical compositions of the invention that contain an enriched population of Hox11+ stem cells may be formulated for parenteral administration, subcutaneous administration, intravenous administration, intramuscular administration, intra-arterial administration, intrathecal administration, or interperitoneal administration (intravenous administration is particularly suitable). The pharmaceutical composition may also be formulated for, or administered via, nasal, spray, oral, aerosol, rectal, or vaginal administration. Methods of administering cells are known in the art. See, for example, U.S. Pat. Nos. 5,423,778, 5,800,828, 6,008,035, 6,306,424, 7,011,828, and 7,031,775, the disclosures of which are incorporated by reference in their entireties. One or more of these methods may be used to administer a pharmaceutical composition of the invention that contains an enriched population of Hox11+ stem cells. In some examples, a pharmaceutical composition of the invention may be delivered directly to an injured tissue or organ. For injectable formulations, various effective pharmaceutical carriers are known in the art. See, e.g., Pharmaceutics and Pharmacy Practice, J. B. Lippincott Company, Philadelphia, Pa., Banker and Chalmers, eds., pages 238-250 (1982), and ASHP Handbook on Injectable Drugs, Toissel, 4th ed., pages 622-630 (1986).

A pharmaceutical composition of the invention may include a dosage of enriched Hox11+ stem cells, e.g., at least about 500,000 Hox11+ stem cells. In other examples, a pharmaceutical composition includes an amount of Hox11+ stem cells in the range of from 500,000 to 80,000,000 cells (e.g., at least about 1-10×10⁶ Hox11+ stem cells).

Pharmaceutical compositions of the invention that contain an enriched population of Hox11+ stem cells may be administered to a subject in need thereof, for example, one or more times (e.g., 1-10 times or more) daily, weekly, monthly, biannually, annually, or as medically necessary. The pharmaceutical composition may be administered on the same day, or within 1-30 days (e.g., within 10 days) or more after the cells are harvested from a donor. The pharmaceutical composition may also be administered at a time relative to the time at which an injury occurs (e.g., immediately after an injury occurs). The timing between administrations may decrease as the medical condition improves or increase as the health of the patient declines.

### Methods of Treatment Using Enriched Hox11+ Stem Cells

The invention provides pharmaceutical compositions containing an enriched population of Hox11+ stem cells that may be used to treat patients in need of, or who have undergone, organ transplantation. Pharmaceutical compositions of the invention may also be used to treat patients in need of, or who have undergone, tissue or organ repair or regeneration. Pharmaceutical compositions of the invention may also be used to treat patients who are undergoing or who have undergone chemotherapy or radiotherapy for cancer.

For patients who are in need of, or who have undergone, organ transplantation, a pharmaceutical composition containing an enriched population of Hox11+ stem cells of the invention may be administered to the patient (e.g., by injection into the patient's bloodstream or directly into or near the organ or site of transplantation). Examples of transplant patients are those that have received a heart, heart valve, blood vessel (e.g., artery or vein), kidney, liver, lung, or lung lobe, pancreas, ovary, bladder, stomach, testis, intestine, thymus, bone, tendon, cornea, skin, nerve, hand, arm, foot, leg, or cellular (e.g., beta-islet cells, stem cells (e.g., hematopoietic stem cells, such as bone marrow stem cells (e.g., CD34+ stem cells)) transplant. The transplant patient may also have received an autologous, allogeneic, or syngeneic cell transplant. The administration of a pharmaceutical composition containing an enriched population of Hox11+ stem cells may provide better engraftment of the transplanted organ, tissue, or cell, or may reduce or eliminate the risk of rejection, e.g., graft-versus-host disease (GVHD).

For patients who are in need of, or who have undergone, tissue or organ repair or regeneration, a pharmaceutical composition containing an enriched population of Hox11+ stem cells of the invention may be administered and may be beneficial in repairing or regenerating damaged or deficient tissues or organs. Examples of such tissues and organs include, e.g., pancreas, salivary gland, pituitary gland, kidney, heart, lung, cranial nerves, heart, blood vessels including the aorta, olfactory gland, ear, nerves, structures of the head, eye, thymus, tongue, bone, liver, small intestine, large intestine, gut, lung, brain, skin, peripheral nervous system, central nervous system, spinal cord, breast, embryonic structures, embryos, and testes.

For patients who are undergoing or who have undergone chemotherapy or radiotherapy, a pharmaceutical composition containing an enriched population of Hox11+ stem cells may be administered to the patient (e.g., by injection into the patient's bloodstream). The administered cells may engraft and restore function to one or more damaged or deficient cells, tissues, or organs.

Furthermore, a pharmaceutical composition of the invention containing an enriched population of Hox11+ stem cells may also be used to treat a medical condition including, but are not limited to, an autoimmune disease, a neurological disorder, cancer, an age-related disease, trauma, and irradiation. For treating one or more of these medical conditions, the pharmaceutical composition may include an amount of Hox11+ stem cells in the range of from about 500,000 to about 80,000,000. Generally, the pharmaceutical composition contains at least about 1-10×10⁶ Hox11+ stem cells, which can be administered to a patient in need thereof. If desired, the patient may be administered at least about 1-10×10⁶ Hox11+ stem cells/kg of body weight.

Exemplary autoimmune diseases that can be treated using an enriched Hox11+ stem cell-containing composition of the invention include Alopecia Areata, Ankylosing Spondylitis, Antiphospholipid Syndrome, Addison's Disease, Hemolytic Anemia, Hepatitis, Behcets Disease, Bullous Pemphigoid, Cardiomyopathy, Celiac Sprue-Dermatitis, Chronic Fatigue Immune Dysfunction Syndrome (CFIDS), Chronic Inflammatory Demyelinating Polyneuropathy, Churg-Strauss Syndrome, Cicatricial Pemphigoid, Limited Scleroderma (CREST Syndrome), Cold Agglutinin Disease, Crohn's Disease, Discoid Lupus, Essential Mixed Cryoglobulinemia, Fibromyalgia-Fibromyositis, Graves' Disease, Guillain-Barré Syndrome, Hashimoto's Thyroiditis, Hypothyroidism, Idiopathic Pulmonary Fibrosis, Idiopathic Thrombocytopenia Purpura (ITP), IgA Nephropathy, Insulin dependent Diabetes, Juvenile Arthritis, Lichen Planus, Lupus, Ménière's Disease, Mixed Connective Tissue Disease, Multiple Sclerosis, Myasthenia Gravis, Pemphigus Vulgaris, Pernicious Anemia, Polyarteritis Nodosa, Polychondritis, Polyglandular Syndromes, Polymyalgia Rheumatica, Polymyositis and Dermatomyositis, Primary Agammaglobulinemia, Primary Biliary Cirrhosis, Psoriasis, Raynaud's Phenomenon, Reiter's Syndrome, Rheumatic Fever, Rheumatoid Arthritis, Sarcoidosis, Scleroderma, Sjögren's Syndrome, Stiff-Man Syndrome, Takayasu Arteritis, Temporal Arteritis/Giant Cell Arteritis, Ulcerative Colitis, Uveitis, Vasculitis, Vitiligo, and Wegener's Granulomatosis. Preferably, the autoimmune disease is insulin dependent diabetes (also known as type 1 diabetes or autoimmune diabetes), multiple sclerosis, rheumatoid arthritis, Crohn's disease, thyroiditis, lupus, Sjögren's Syndrome, and dermatitis. In particular methods, a patient to be treated according to the invention has type 1 diabetes.

Exemplary neurological disorders that can be treated using an enriched Hox11+ stem cell-containing composition of the invention include Parkinson's disease, Alzheimer's disease, Amyotrophic lateral sclerosis (ALS), Huntington's disease, traumatic brain injury, and spinal cord injury.

Exemplary cancers that can be treated using an enriched Hox11+ stem cell-containing composition of the invention include bladder cancer, pancreatic cancer, cervical cancer, lung cancer, liver cancer, ovarian cancer, colon cancer, stomach cancer, virally induced cancer, neuroblastoma, breast cancer, prostate cancer, renal cancer, leukemia, sarcomas, and carcinomas.

Exemplary age-related diseases that can be treated using an enriched Hox11+ stem cell-containing composition of the invention include metabolic disorder, inflammatory disorder, cardiovascular disease, diabetes type 1, diabetes type 2, artherosclerosis, Alzheimer's disease, dementia, clinical depression, obesity, muscular dystrophy, sarcopenia, cachexia and osteoporosis.

### EXAMPLES

### Example 1—Collection of Hox11+ Stem Cells from Mobilized Peripheral Blood

### Materials

All −70° C. frozen human peripheral blood cell samples used for this study were either from the Core Center of Excellence in Hematology (CCEH) at the Fred Hutchison Cancer Research Center or from Massachusetts General Hospital (MGH). These peripheral blood samples were obtained from donors not treated (non-mobilized samples) or treated with recombinant G-CSF (mobilized samples). Additional samples include peripheral blood samples from G-CSF treated donors that were further enriched for CD34+ stem cells. The non-mobilized, mobilized, and CD34+ stem cell enriched samples were obtained following human consent protocol 985.03-2 from Fred Hutchison Cancer Research Center. Additional fresh peripheral blood lymphocytes (PBLs) that were used to standardize the mRNA expressions of the frozen PBLs samples were obtained following human consent protocol MGH-2001P001379 from MGH, which involved the obligatory informed consent of all subjects.

### Methods

Peripheral blood lymphocytes (PBLs) isolated from peripheral blood samples through leukapheresis were thawed in 37° C. water bath and washed with PBS+1% FBS twice. To measure the mRNA expressions of CD34 and Hox11 genes, total RNA was extracted using the RNeasy Mini Kit (QIAGEN, Valencia, Calif.) according to the manufacturer's instructions. Complementary DNA (cDNA) was generated using the High Capacity cDNA Reverse Transcription Kit (Applied Biosystems, Foster City, Calif.). Quantitative real-time PCR was performed using Power SYBR Green reagent (Applied Biosystems, Foster City, Calif.) and the 7000 Real-Time PCR System.

Relative mRNA expression was calculated using a comparative count method (ddCT method). The mRNA expression of β-actin gene was used as a control to normalize the data. The database of National Center for Biotechnology Information was used as a resource. Specific primer constructs were designed as shown below.

Human Hox11/Tlx1 gene (Gene ID: 3195) primers:
forward sequence: 5′- GGTTCACAGGTCACCCCTATC -3′
reverse sequence: 5′- GTCTGCCGTCTCCACTTTGTC -3′
CD34 gene (Gene ID: 947) primers
forward sequence: 5′-CTACAACACCTAGTACCCTTGGA-3′
reverse sequence: 5′- GGTGAACACTGTGCTGATTACA-3′
β-actin gene (Gene ID: 60) primers
forward sequence: 5′-CATGTACGTTGCTATCCAGGC-3′
reverse sequence: 5′-CTCCTTAATGTCACGCACGAT-3′

All primers were purchased from Custom DNA Oligos (Invitrogen, St. Louis, Mo.). The cell line ALL-SIL (DSMZ, Braunschweig, Germany) that expressed Hox11/Tlx1 was used as a positive control to ensure that the primers for the Hox11/Tlx1 gene work well on a Hox11/Tlx1-expressing cell line. All data analyses were performed by the paired Student t test using GraphPad Prism-5 software (GraphPad Software, Inc., La Jolla, Calif.).

### Example 2—G-CSF Mobilized Peripheral Blood Contains CD34+ Stem Cells and Hox11 Stem Cells

Ten human donors not treated with G-CSF provided non-mobilized peripheral blood samples and eighteen donors treated with G-CSF provided mobilized peripheral blood samples. All human donors were healthy and without underlying malignancies. Peripheral blood lymphocytes (PBLs) were isolated from either non-mobilized or mobilized peripheral blood samples using leukapheresis.

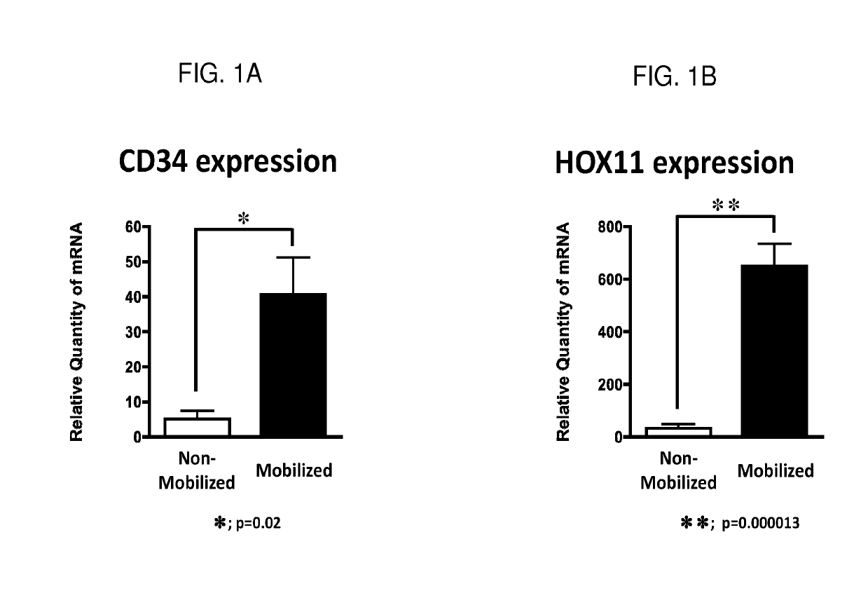

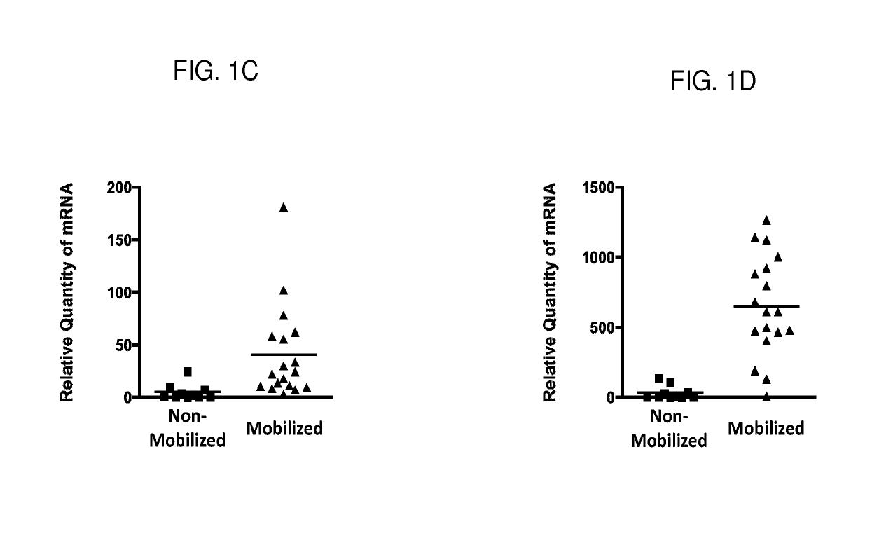

Following the method described above, the expressions of CD34 and Hox11 genes were studied using quantitative mRNA. The data in FIGS. 1 (A) and (C) show that CD34 mRNA expression was exclusively found in PBLs isolated from mobilized peripheral blood samples that were obtained from donors treated with G-CSF (p=0.02). CD34+ stem cells are generally not observed in PBLs isolated from non-mobilized peripheral blood samples that were obtained from donors not treated with G-CSF. The data in these samples confirm the benefit of G-CSF in mobilizing stem cells that reside in the bone marrow, e.g., CD34+ stem cells, into the peripheral blood.

Comparing Hox11 gene expression in PBLs obtained from non-mobilized and mobilized peripheral blood samples, the data in FIGS. 1 (B) and (D) show that Hox11 mRNA expression was also exclusively found in PBLs isolated from mobilized peripheral blood samples that were obtained from donors treated with G-CSF (p=0.000013). The data confirm that the administration of G-CSF was able to mobilize Hox11+ stem cells residing in the spleen, in addition to CD34+ stem cells residing in the bone marrow, to the peripheral blood.

### Example 3—CD34+ Stem Cells and Hox11+ Stem Cells in G-CSF Mobilized Peripheral Blood are Distinct Stem Cell Populations

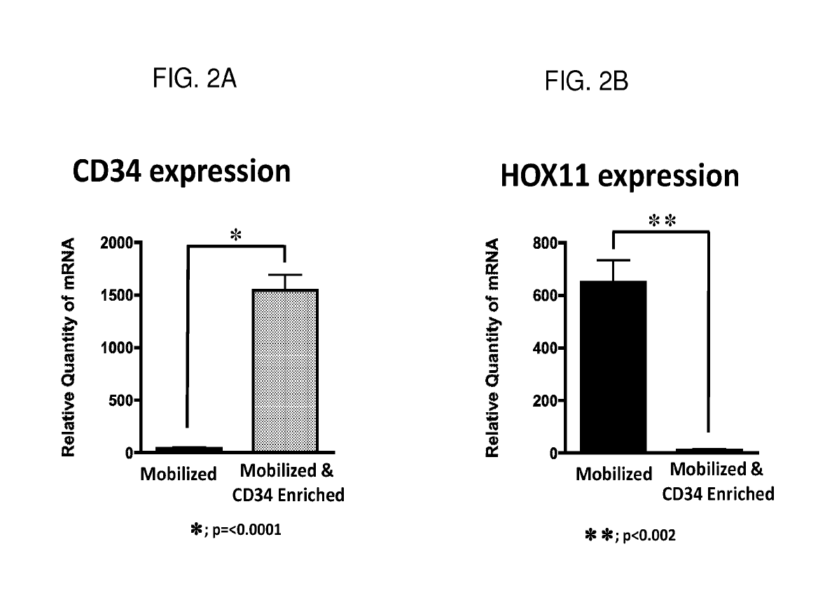

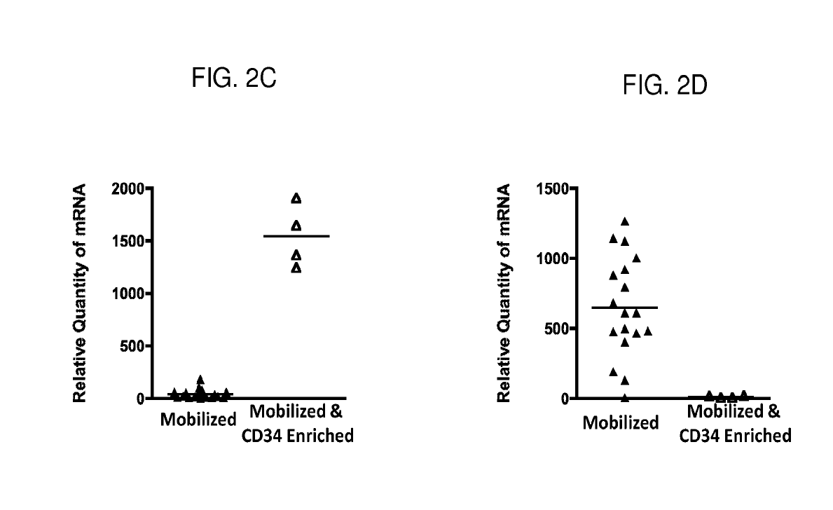

To ensure that the CD34+ stem cells and the Hox11+ stem cells detected in the PBLs of mobilized peripheral blood (Example 2) are indeed two different populations of stem cells and not caused by aberrant expression of Hox11 gene by CD34+ stem cells, PBLs obtained from mobilized peripheral blood samples were specifically enriched for CD34+ stem cells using positive magnetic bead enrichment. Using quantitative mRNA expression analysis, data in FIGS. 2 (A) and (C) show the expected, strong expression of CD34 in the G-CSF mobilized, CD34+ stem cell enriched PBLs (p=<0.0001). Additionally, data in FIGS. 2 (B) and (D) effectively demonstrate the lack of Hox11 expression in the same G-CSF mobilized, CD34+ stem cell enriched PBLs (p<0.002). Therefore, FIG. 2 shows that G-CSF treatment can mobilize at least two distinct populations of stem cells, CD34+ stem cells and Hox11+ stem cells.

### OTHER EMBODIMENTS

All publications, patents, and patent applications mentioned in the above specification are hereby incorporated by reference. Various modifications and variations of the described compositions and methods of use of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are within the following claims.

1. A method of preparing a pharmaceutical composition, comprising:
a) administering at least one mobilization agent to a subject; and
b) preparing said pharmaceutical composition by collecting Hox11+ stem cells from peripheral blood of the subject,
wherein the composition comprises a cellular component having at least 1% Hox11+ stem cells.

2. The method of claim 1, wherein said mobilization agent is selected from the group consisting of granulocyte colony-stimulating factor (G-CSF), granulocyte-macrophage colony-stimulating factor (GM-CSF), stem cell factor (SCF), Fms-related tyrosine kinase 3 (flt-3) ligand, stromal cell-derived factor 1 (SDF-1), agonists of the chemokine (C—C motif) receptor 1 (CCR1), such as chemokine (C—C motif) ligand 3 (CCL3, also known as macrophage inflammatory protein-1α (Mip-1α)), agonists of the chemokine (C—X—C motif) receptor 1 (CXCR1) and CXCR2, such as chemokine (C—X—C motif) ligand (CXCL1), CXCL2 (also known as growth-related oncogene protein-β (Gro-β)), and CXCL8 (also known as interleukin-8 (IL-8)), agonists of CXCR4, such as CTCE-002, ATI-2341, and Met-SDF-1, Very Late Antigen (VLA)-4 inhibitor, TG-0054, plerixafor (also known as AMD3100), AMD3465, and any combination thereof.

3. The method of claim 2, wherein said mobilization agent is G-CSF.

4. The method of claim 1, wherein said mobilization agent is administered in combination with one or more chemotherapy agents or immunostimulants.

5. The method of claim 4, wherein said G-CSF is administered in combination with an immunostimulant, particularly wherein said immunostimulant is plerixafor.

6. The method of claim 1, wherein said preparing comprises apheresis, such as leukapheresis.

7. The method of claim 1, wherein said preparing comprises removing non-Hox11+ stem cells from said composition, such as by use of an antibody.

8. The method of claim 1, wherein said preparing comprises enriching for Hox11+ stem cells by removal of CD45+ cells.

9. The method of claim 1, wherein said preparing comprises enriching for Hox11 expressing stem cells by removal of CD34+ stem cells.

10. The method of claim 7, wherein said antibody is attached to a magnetic bead and said removing comprises separating non-Hox11+ stem cells from said composition using magnets, or said antibody is attached to a fluorophore and said removing comprises separating non-Hox11+ stem cells from said composition using fluorescence-activated cell sorting (FACS).

11. The method of claim 7 or 10, wherein said non-Hox11-stem cells are characterized by expression of one or more of cell-surface markers selected from the group consisting of CD3, CD4, CD16, CD19, CD20, CD21, CD34, CD45, CD56, and T cell receptor.

12. The method of claim 1, further comprising quantifying the number of Hox11+ stem cells in said composition, particularly wherein said quantifying comprises detecting the number of Hox11+ cells in said composition relative to the number of non-Hox11+ cells in said composition.

13. The method of claim 12, wherein said Hox11+ stem cells are detected using an intracellular protein or mRNA marker, particularly wherein said intracellular protein or mRNA marker is selected from the group consisting of Hox11, Mad2L1, Minichromosome maintenance complex component 7 (Mcm7), Mcm8, POLD1, Hox11, DNA topoisomerase 1 (Top1), and Top2β.

14. The method of claim 12, wherein said non-Hox11+ cells are detected using a protein or mRNA marker that is not present on Hox11+ stem cells, such as by use of an antibody or by nucleic acid amplification, particularly wherein said marker is selected from one or more of the following Dhfr, Ercc1, Hprt1, Lap18, Mad1/1, Pola, Polr2e, Tdt, Topbp1, and Ung or a surface marker of mature lymphocytes selected from one or more of CD3, CD4, CD16, CD19, CD20, CD21, CD34, CD45, CD56, and T cell receptor.

15. The method of claim 12, wherein said quantifying comprises using quantitative polymerase chain reaction (PCR), particularly wherein said quantifying comprises detecting Hox11+ stem cells using primers specific to the Hox11 gene.

16. The method of claim 1, wherein said composition comprises Hox11+ stem cells and CD34+ stem cells, wherein the ratio of said Hox11+ stem cells to said CD34+ stem cells is 9:1 to 1:9.

17. The method of claim 1, wherein the cellular component comprises about 1-90% Hox11+ stem cells, particularly wherein the cellular component comprises at least about 5%, 10%, 20%, 25%, 30%, 35%, 40%, 45%, 50%, 55%, 60%, 65%, 70%, 75%, 80%, or 85% Hox11+ stem cells.

18. The method of claim 17, wherein at least 25% of the cells in the composition are Hox11+ stem cells.

19. The method of claim 18, wherein at least 50% of the cells in the composition are Hox11+ stem cells.

20. The method of claim 19, wherein at least 75% of the cells in the composition are Hox11+ stem cells.

21. The method of claim 1, wherein said cellular component of the composition comprises a substantially homogeneous Hox11+ stem cell population.

22. A pharmaceutical composition comprising a cell population that comprises at least 1% Hox11+ stem cells.

23. The pharmaceutical composition of claim 22, wherein said population comprises about 1-90% Hox11+ stem cells, particularly wherein the composition comprises about 5%, 10%, 20%, 25%, 30%, 35%, 40%, 45%, 50%, 55%, 60%, 65%, 70%, 75%, 80%, or 85% Hox11+ stem cells.

24. The pharmaceutical composition of claim 22 comprising Hox11+ stem cells and CD34+ stem cells, wherein the ratio of said Hox11+ stem cells to said CD34+ stem cells is 9:1 to 1:9.

25. The pharmaceutical composition of claim 22, wherein said composition is made by the method of claim 1.

26. The pharmaceutical composition of claim 22, wherein said composition comprises one or more pharmaceutically acceptable carriers or excipients.

27. A method of medical therapy comprising administering the pharmaceutical composition of claim 22 to a subject in need thereof.

28. The method of claim 27, wherein said subject is in need of tissue or organ repair or regeneration.

29. The method of claim 28, wherein said tissue or organ is pancreas, salivary gland, pituitary gland, kidney, heart, lung, hematopoietic system, cranial nerves, heart, blood vessels including the aorta, olfactory gland, ear, nerves, structures of the head, eye, thymus, tongue, bone, liver, small intestine, large intestine, gut, lung, brain, skin, peripheral nervous system, central nervous system, spinal cord, breast, embryonic structures, embryos, or testes.

30. The method of claim 28 or 29, wherein said tissue or organ is damaged or deficient.

31. The method of claim 27, wherein said subject has an autoimmune disease, a neurological disorder, cancer, an age-related disease, trauma, or acute radiation syndrome.

32. The method of claim 31, wherein said autoimmune disease is selected from Alopecia Areata, Ankylosing Spondylitis, Antiphospholipid Syndrome, Addison's Disease, Hemolytic Anemia, Hepatitis, Behcets Disease, Bullous Pemphigoid, Cardiomyopathy, Celiac Sprue-Dermatitis, Chronic Fatigue Immune Dysfunction Syndrome (CFIDS), Chronic Inflammatory Demyelinating Polyneuropathy, Churg-Strauss Syndrome, Cicatricial Pemphigoid, Limited Scleroderma (CREST Syndrome), Cold Agglutinin Disease, Crohn's Disease, Discoid Lupus, Essential Mixed Cryoglobulinemia, Fibromyalgia-Fibromyositis, Graves' Disease, Guillain-Barré Syndrome, Hashimoto's Thyroiditis, Hypothyroidism, Idiopathic Pulmonary Fibrosis, Idiopathic Thrombocytopenia Purpura (ITP), IgA Nephropathy, Insulin dependent Diabetes, Juvenile Arthritis, Lichen Planus, Lupus, Ménière's Disease, Mixed Connective Tissue Disease, Multiple Sclerosis, Myasthenia Gravis, Pemphigus Vulgaris, Pernicious Anemia, Polyarteritis Nodosa, Polychondritis, Polyglandular Syndromes, Polymyalgia Rheumatica, Polymyositis and Dermatomyositis, Primary Agammaglobulinemia, Primary Biliary Cirrhosis, Psoriasis, Raynaud's Phenomenon, Reiter's Syndrome, Rheumatic Fever, Rheumatoid Arthritis, Sarcoidosis, Scleroderma, Sjögren's Syndrome, Stiff-Man Syndrome, Takayasu Arteritis, Temporal Arteritis/Giant Cell Arteritis, Ulcerative Colitis, Uveitis, Vasculitis, Vitiligo, and Wegener's Granulomatosis, particularly wherein said autoimmune disease is Insulin dependent Diabetes.

33. The method of claim 31, wherein said neurological disorder is selected from Parkinson's disease, Alzheimer's disease, Amyotrophic lateral sclerosis (ALS), Huntington's disease, traumatic brain injury, and spinal cord injury.

34. The method of claim 31, wherein said cancer is selected from bladder cancer, pancreatic cancer, cervical cancer, lung cancer, liver cancer, ovarian cancer, colon cancer, stomach cancer, virally induced cancer, neuroblastoma, breast cancer, prostate cancer, renal cancer, leukemia, sarcoma, and carcinoma.

35. The method of claim 31, wherein said age-related disease is selected from a metabolic disorder, an inflammatory disorder, a cardiovascular disease, diabetes type 1, diabetes type 2, artherosclerosis, Alzheimer's disease, dementia, clinical depression, obesity, muscular dystrophy, sarcopenia, cachexia and osteoporosis.

36. The method of claim 27, wherein said subject is in need or, or has received, a cellular, tissue, or organ transplant.

37. The method of claim 36, wherein said transplant is a heart, heart valve, blood vessel (e.g., artery or vein), kidney, liver, lung, or lung lobe, pancreas, ovary, bladder, stomach, testis, intestine, thymus, bone, tendon, cornea, skin, nerve, hand, arm, foot, leg, beta-islet cell, or stem cell transplant.

38. The method of claim 27, wherein the Hox11+ stem cells in said composition are allogeneic or autologous to said subject.

39. A method of preparing a pharmaceutical composition, comprising preparing said pharmaceutical composition by collecting Hox11+ stem cells from peripheral blood of a subject administered at least one mobilization agent, wherein the composition comprises a cellular component having at least 1% Hox11+ stem cells.

40. The method of any one of claims 1 to 3, wherein said mobilization agent is administered in combination with one or more chemotherapy agents or immunostimulants.

41. The method of claim 40, wherein said G-CSF is administered in combination with an immunostimulant, particularly wherein said immunostimulant is plerixafor.

42. The method of any one of claims 1 to 3, 40, and 41, wherein said preparing comprises apheresis, such as leukapheresis.

43. The method of any one of claims 1 to 3 and 40 to 42, wherein said preparing comprises removing non-Hox11+ stem cells from said composition, such as by use of an antibody.

44. The method of any one of claims 1 to 3 and 40 to 43, wherein said preparing comprises enriching for Hox11+ stem cells by removal of CD45+ cells.

45. The method of any one of claims 1 to 3 and 40 to 44, wherein said preparing comprises enriching for Hox11 expressing stem cells by removal of CD34+ stem cells.

46. The method of claim 43, wherein said antibody is attached to a magnetic bead and said removing comprises separating non-Hox11+ stem cells from said composition using magnets, or said antibody is attached to a fluorophore and said removing comprises separating non-Hox11+ stem cells from said composition using fluorescence-activated cell sorting (FACS).

47. The method of claim 43 or 46, wherein said non-Hox11-stem cells are characterized by expression of one or more of cell-surface markers selected from the group consisting of CD3, CD4, CD16, CD19, CD20, CD21, CD34, CD45, CD56, and T cell receptor.

48. The method of any one of claims 1 to 3 and 40 to 47, further comprising quantifying the number of Hox11+ stem cells in said composition, particularly wherein said quantifying comprises detecting the number of Hox11+ cells in said composition relative to the number of non-Hox11+ cells in said composition.

49. The method of claim 48, wherein said Hox11+ stem cells are detected using an intracellular protein or mRNA marker, particularly wherein said intracellular protein or mRNA marker is selected from the group consisting of Hox11, Mad2L1, Minichromosome maintenance complex component 7 (Mcm7), Mcm8, POLD1, Hox11, DNA topoisomerase 1 (Top1), and Top2β.

50. The method of claim 48, wherein said non-Hox11+ cells are detected using a protein or mRNA marker that is not present on Hox11+ stem cells, such as by use of an antibody or by nucleic acid amplification, particularly wherein said marker is selected from one or more of the following Dhfr, Ercc1, Hprt1, Lap18, Mad1/1, Pola, Polr2e, Tdt, Topbp1, and Ung or a surface marker of mature lymphocytes selected from one or more of CD3, CD4, CD16, CD19, CD20, CD21, CD34, CD45, CD56, and T cell receptor.

51. The method of claim 48, wherein said quantifying comprises using quantitative polymerase chain reaction (PCR), particularly wherein said quantifying comprises detecting Hox11+ stem cells using primers specific to the Hox11 gene.

52. The method of any one of claims 1 to 3, 40 to 44, and 46 to 51, wherein said composition comprises Hox11+ stem cells and CD34+ stem cells, wherein the ratio of said Hox11+ stem cells to said CD34+ stem cells is 9:1 to 1:9.

53. The method of any one of claims 1 to 3 and 40 to 52, wherein the cellular component comprises about 1-90% Hox11+ stem cells, particularly wherein the cellular component comprises at least about 5%, 10%, 20%, 25%, 30%, 35%, 40%, 45%, 50%, 55%, 60%, 65%, 70%, 75%, 80%, or 85% Hox11+ stem cells.

54. The method of claim 53, wherein at least 25% of the cells in the composition are Hox11+ stem cells.

55. The method of claim 54, wherein at least 50% of the cells in the composition are Hox11+ stem cells.

56. The method of claim 55, wherein at least 75% of the cells in the composition are Hox11+ stem cells.

57. The method of any one of claims 1 to 3 and 40 to 56, wherein said cellular component of the composition comprises a substantially homogeneous Hox11+ stem cell population.

58. The pharmaceutical composition of any one of claims 22 to 24, wherein said composition is made by the method of any one of claims 1 to 21.

59. The pharmaceutical composition of any one of claims 22 to 24 and 58, wherein said composition comprises one or more pharmaceutically acceptable carriers or excipients.

60. A method of medical therapy comprising administering the pharmaceutical composition of any one of claims 22 to 24, 58, and 59 to a subject in need thereof.

61. The method of claim 60, wherein said subject is in need of tissue or organ repair or regeneration.

62. The method of claim 61, wherein said tissue or organ is pancreas, salivary gland, pituitary gland, kidney, heart, lung, hematopoietic system, cranial nerves, heart, blood vessels including the aorta, olfactory gland, ear, nerves, structures of the head, eye, thymus, tongue, bone, liver, small intestine, large intestine, gut, lung, brain, skin, peripheral nervous system, central nervous system, spinal cord, breast, embryonic structures, embryos, or testes.

63. The method of claim 61 or 62, wherein said tissue or organ is damaged or deficient.

64. The method of claim 60, wherein said subject has an autoimmune disease, a neurological disorder, cancer, an age-related disease, trauma, or acute radiation syndrome.

65. The method of claim 64, wherein said autoimmune disease is selected from Alopecia Areata, Ankylosing Spondylitis, Antiphospholipid Syndrome, Addison's Disease, Hemolytic Anemia, Hepatitis, Behcets Disease, Bullous Pemphigoid, Cardiomyopathy, Celiac Sprue-Dermatitis, Chronic Fatigue Immune Dysfunction Syndrome (CFIDS), Chronic Inflammatory Demyelinating Polyneuropathy, Churg-Strauss Syndrome, Cicatricial Pemphigoid, Limited Scleroderma (CREST Syndrome), Cold Agglutinin Disease, Crohn's Disease, Discoid Lupus, Essential Mixed Cryoglobulinemia, Fibromyalgia-Fibromyositis, Graves' Disease, Guillain-Barré Syndrome, Hashimoto's Thyroiditis, Hypothyroidism, Idiopathic Pulmonary Fibrosis, Idiopathic Thrombocytopenia Purpura (ITP), IgA Nephropathy, Insulin dependent Diabetes, Juvenile Arthritis, Lichen Planus, Lupus, Ménière's Disease, Mixed Connective Tissue Disease, Multiple Sclerosis, Myasthenia Gravis, Pemphigus Vulgaris, Pernicious Anemia, Polyarteritis Nodosa, Polychondritis, Polyglandular Syndromes, Polymyalgia Rheumatica, Polymyositis and Dermatomyositis, Primary Agammaglobulinemia, Primary Biliary Cirrhosis, Psoriasis, Raynaud's Phenomenon, Reiter's Syndrome, Rheumatic Fever, Rheumatoid Arthritis, Sarcoidosis, Scleroderma, Sjögren's Syndrome, Stiff-Man Syndrome, Takayasu Arteritis, Temporal Arteritis/Giant Cell Arteritis, Ulcerative Colitis, Uveitis, Vasculitis, Vitiligo, and Wegener's Granulomatosis, particularly wherein said autoimmune disease is Insulin dependent Diabetes.

66. The method of claim 64, wherein said neurological disorder is selected from Parkinson's disease, Alzheimer's disease, Amyotrophic lateral sclerosis (ALS), Huntington's disease, traumatic brain injury, and spinal cord injury.

67. The method of claim 64, wherein said cancer is selected from bladder cancer, pancreatic cancer, cervical cancer, lung cancer, liver cancer, ovarian cancer, colon cancer, stomach cancer, virally induced cancer, neuroblastoma, breast cancer, prostate cancer, renal cancer, leukemia, sarcoma, and carcinoma.

68. The method of claim 64, wherein said age-related disease is selected from a metabolic disorder, an inflammatory disorder, a cardiovascular disease, diabetes type 1, diabetes type 2, artherosclerosis, Alzheimer's disease, dementia, clinical depression, obesity, muscular dystrophy, sarcopenia, cachexia and osteoporosis.

69. The method of claim 60, wherein said subject is in need or, or has received, a cellular, tissue, or organ transplant.

70. The method of claim 69, wherein said transplant is a heart, heart valve, blood vessel (e.g., artery or vein), kidney, liver, lung, or lung lobe, pancreas, ovary, bladder, stomach, testis, intestine, thymus, bone, tendon, cornea, skin, nerve, hand, arm, foot, leg, beta-islet cell, or stem cell transplant.

71. The method of any one of claims 60-70, wherein the Hox11+ stem cells in said composition are allogeneic or autologous to said subject.


SEQUENCE LISTING (6 sequences)

SEQ ID NO: 1 - DNA, 21 bp, Homo sapiens
ggttcacaggtcacccctatc

SEQ ID NO: 2 - DNA, 21 bp, Homo sapiens
gtctgccgtctccactttgtc

SEQ ID NO: 3 - DNA, 23 bp, Homo sapiens
ctacaacacctagtacccttgga

SEQ ID NO: 4 - DNA, 22 bp, Homo sapiens
ggtgaacactgtgctgattaca

SEQ ID NO: 5 - DNA, 21 bp, Homo sapiens
catgtacgttgctatccaggc

SEQ ID NO: 6 - DNA, 21 bp, Homo sapiens
ctccttaatgtcacgcacgat